(19) United States
(12) Patent Application Publication
(10) Pub. No.: US 2021/0298657 A1
(43) Pub. Date: Sep. 30, 2021

(54) Wireless Sound Converting System Having Bio-Signal Sensing Function

(71) Applicant: EM-TECH Co., Ltd., Gyeongsangnam-do (KR)

(72) Inventor: Hyuk Won, Busan (KR)

(21) Appl. No.: 17/210,764

(22) Filed: Mar. 24, 2021

(30) Foreign Application Priority Data
Mar. 27, 2020 (KR) 10-2020-0037438

(51) Int. Cl. A61B 5/33; A61B 5/00; A61B 5/11; H04R 1/02

(52) U.S. Cl. CPC A61B 5/33; A61B 5/6815; A61B 5/0059; H04R 2420/07; A61B 5/1126; H04R 1/02; A61B 2562/0219; A61B 5/7405

(57) ABSTRACT

At least one of first and second wireless sound converting devices of a wireless sound converting system includes: an internal electrode unit mounted on a first side of a body portion and configured to detect electrocardiogram (ECG), and an external electrode unit mounted on the opposite side or side adjacent the opposite side and including an ECG sensor configured to apply a first detection value related to ECG to a data processor and a photoplethysmography (PPG) sensor mounted on the first side of the body portion and configured to detect PPG and apply a second detection value related to PPG to the data processor. With the wireless sound converting device that includes the ECG sensor mounted on a wearer's ear, the corresponding data processor is configured to emit sound of a message for configuring a bio-signal transmission path using the wearer's body through the speaker to measure ECG.

### TECHNICAL FIELD

The present disclosure relates to a wireless sound converting system, and in particular, to a wireless sound converting system having a bio-signal detection function to detect and process a bio-signal from an ear of a head with relatively little movement.

### BACKGROUND

Wireless sound converting devices include sound devices such as earphones, earbuds, and headsets and play sound or make a phone call, while performing wireless communication with electronic devices (e.g., smartphones, tablets, etc.).

However, the related art wireless sound converting device does not provide a function for health maintenance or a treatment other than sound reproduction or phone calls to wearers.

### SUMMARY

An aspect of the present disclosure provides a wireless sound converting system having a bio-signal detection function to more accurately detect or measure and process a bio-signal in an ear of a head with relatively little movement.

According to an aspect of the present disclosure, there is provided a wireless sound converting system having a bio-signal detection function including first and second wireless sound converting devices including a body portion having an accommodation space formed therein, an ear tip mounted on a first side of the body portion and having a sound passage, a speaker emitting sound through a sound path, a microphone formed in the body portion, a communication unit performing communication with an electronic communication device, and a data processor performing a sound reproduction function by controlling the communication unit to communicate with the electronic communication device, wherein at least one of the first and second wireless sound converting devices includes: an internal electrode unit mounted on the first side of the body portion to detect electrocardiogram (ECG) and an external electrode unit mounted on a side opposite to the first side or a side adjacent to the opposite side and includes an ECG sensor applying a first detection value related to ECG to the data processor and a photoplethysmography (PPG) sensor mounted on the first side of the body portion to detect PPG and applying a second detection value related to PPG to the data processor, wherein in a state in which the first or second wireless sound converting device including the ECG sensor is mounted on a wearer's ear, the data processor of the mounted wireless sound converting device emits sound of a message for configuring a bio-signal transmission path using the wearer's body through the speaker to measure ECG.

In addition, at least one wireless sound converting device including the PPG sensor, among the first and second wireless sound converting devices, may include an inertial measurement unit (IMU) sensor applying a third detection value for a change in inertia to the data processor.

In addition, the data processor of the mounted wireless sound converting device may emit, through the speaker, sound of a message for configuring a bio-signal transmission path that allows the wearer to press and contact the outer electrode unit with a hand or a finger.

In addition, any one or more of the data processors of the first and second wireless sound converting devices may transmit at least one of the first to third detection values to the electronic communication device through the communication unit or determine a physical condition of the wearer who wears the first or second wireless sound converting device using at least one of the first detection value and the second detection value, and notify the determined physical condition through the speaker.

In addition, any one or more of the data processors of the first and second wireless sound converting devices may determine the physical condition of the wearer using the second detection value and the third detection value.

In addition, the wireless sound converting system may include an electronic communication device receiving at least one or more of the first to third detection values from at least one or more of the first and second wireless sound converting devices, and the electronic communication device may determine the wearer's physical condition using at least one or more of the first to third detection values, and transmit the determined physical condition to at least one or more of the first and second wireless sound converting devices.

In the present disclosure, a bio-signal is detected and processed in an ear of a head with relatively little movement, and in a normal node, a physical condition of a wearer may be determined using a photoplethysmography (PPG) detection value, and if the physical condition of the wearer is abnormal in the normal mode, a bio-signal transmission path is guided for electrocardiogram (ECG) detection in a precision mode and the physical condition of the wearer is more accurately determined using the PPG detection value, an ECG detection value, and a change in inertia, whereby the physical condition of the wearer may be determined and informed using a bio-signal.

Those skilled in the art will recognize additional features and advantages upon reading the following detailed description, and upon viewing the accompanying drawings.

### BRIEF DESCRIPTION OF THE DRAWINGS

FIGS. 1A and 1B are a front view and a side perspective view, respectively, of a wireless sound converting device included in a wireless sound converting system having a bio-signal detection function according to the present disclosure.

FIG. 2 is a block diagram of a wireless sound converting system having a bio-signal detection function according to the present disclosure.

FIG. 3 is a schematic diagram of configuring a bio-signal transmission path for detecting a first detection value of an ECG sensor.

### DETAILED DESCRIPTION

Hereinafter, various embodiments of the present disclosure will be described with reference to the accompanying drawings. Accordingly, those of ordinary skill in the art will recognize that modification, equivalent, and/or alternative on the various embodiments described herein may be variously made without departing from the scope and spirit of the present disclosure. With regard to description of drawings, similar components may be marked by similar reference numerals.

In the disclosure disclosed herein, the expressions “have”, “may have”, “include” and “comprise”, or “may include” and “may comprise” used herein indicate existence of corresponding features (e.g., elements such as numeric values, functions, operations, or components) but do not exclude presence of additional features.

In the disclosure disclosed herein, the expressions “A or B”, “at least one of A or/and B”, or “one or more of A or/and B”, and the like used herein may include any and all combinations of one or more of the associated listed items. For example, the term “A or B”, “at least one of A and B”, or “at least one of A or B” may refer to all of the case (1) where at least one A is included, the case (2) where at least one B is included, or the case (3) where both of at least one A and at least one B are included.

The terms, such as “first”, “second”, and the like used herein may refer to various elements of various embodiments of the present disclosure, but do not limit the elements. For example, such terms are used only to distinguish an element from another element and do not limit the order and/or priority of the elements. For example, a first user device and a second user device may represent different user devices irrespective of sequence or importance. For example, without departing the scope of the present disclosure, a first element may be referred to as a second element, and similarly, a second element may be referred to as a first element.

It will be understood that when an element (e.g., a first element) is referred to as being “(operatively or communicatively) coupled with/to” or “connected to” another element (e.g., a second element), it may be directly coupled with/to or connected to the other element or an intervening element (e.g., a third element) may be present. In contrast, when an element (e.g., a first element) is referred to as being “directly coupled with/to” or “directly connected to” another element (e.g., a second element), it should be understood that there are no intervening element (e.g., a third element).

According to the situation, the expression “configured to” used herein may be used as, for example, the expression “suitable for”, “having the capacity to”, “designed to”, “adapted to”, “made to”, or “capable of”. The term “configured to (or set to)” must not mean only “specifically designed to” in hardware. Instead, the expression “a device configured to” may mean that the device is “capable of” operating together with another device or other components. CPU, for example, a “processor configured to (or set to) perform A, B, and C” may mean a dedicated processor (e.g., an embedded processor) for performing a corresponding operation or a generic-purpose processor (e.g., a central processing unit (CPU) or an application processor) which may perform corresponding operations by executing one or more software programs which are stored in a memory device.

Terms used in this specification are used to describe specified embodiments of the present disclosure and are not intended to limit the scope of the present disclosure. The terms of a singular form may include plural forms unless otherwise specified. Unless otherwise defined herein, all the terms used herein, which include technical or scientific terms, may have the same meaning that is generally understood by a person skilled in the art. It will be further understood that terms, which are defined in a dictionary and commonly used, should also be interpreted as is customary in the relevant related art and not in an idealized or overly formal detect unless expressly so defined herein in various embodiments of the present disclosure. In some cases, even if terms are terms which are defined in the specification, they may not be interpreted to exclude embodiments of the present disclosure.

In the present disclosure, a bio-signal corresponds to a signal for a wearer's living body including an electrocardiogram (ECG) and photoplethysmography (PPG)), etc.

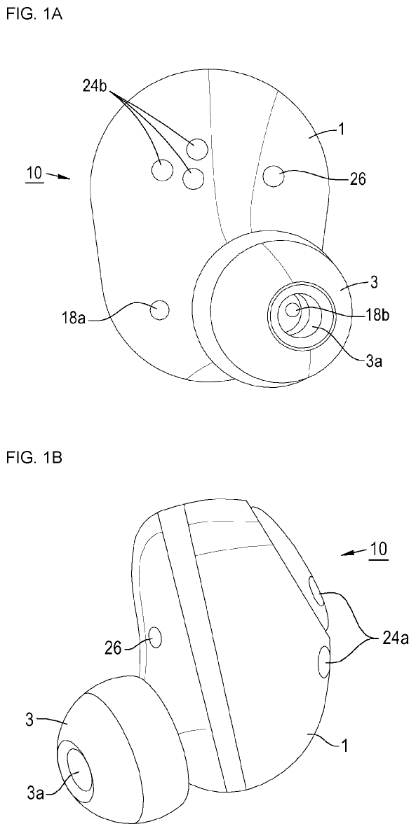

FIGS. 1A and 1B are a front view and a side perspective view, respectively, of a wireless sound converting device of a wireless sound converting system having a bio-signal detection function according to the present disclosure.

A wireless acoustic converting device 10 includes a body portion 1 including a control device such as a circuit board in an accommodation space, an ear tip 3 mounted on a first side of the body portion 1 and having a sound passage 3a, a first optical element 18a mounted on the first side of the body portion 1, a second optical element 18b mounted at the sound passage 3a of the ear tip 3, an electrode unit 24 (24a and 24b) in contact with a skin of a wearer to detect ECG, and a PPG sensor 26 in close proximity to the skin of the wearer to detect PPG.

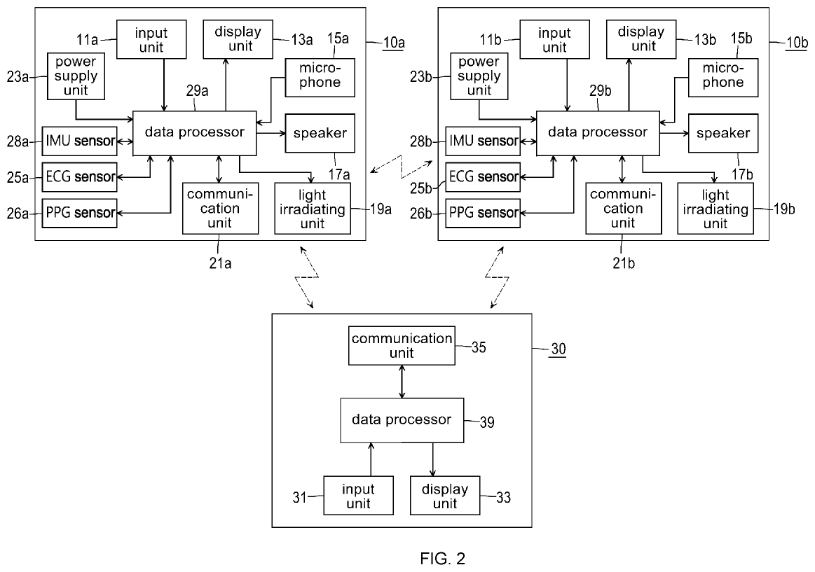

The body portion 1 includes a control device as shown in FIG. 2 in an accommodation space formed therein and is worn on an outer ear (or an auricular concha) when the user wears it, and the ear tip 3 is inserted into the ear (e.g., an ear canal). The side opposite to the first side of the body portion 1 (a side symmetrical to the first side with respect to the body portion 1) faces the outside when the user inserts and mounts the wireless sound converting device 10 in the ear.

At least a portion of the first side of the body portion 1 is in close proximity to or in contact with an ear auricle (e.g., the auricular concha), and an effect based on light (e.g., near-infrared rays) irradiated by the first optical element 18a may be achieved. In addition, the second optical element 18b irradiates light toward a front outside of the ear tip 3 through the acoustic passage 3a and performs light irradiation into the ear in a state in which the ear tip 3 is inserted in the ear canal.

When the body portion 1 is inserted and worn on the outer ear, the ear tip 3 is inserted and seated in the ear.

The electrode unit 24 includes a first electrode unit 24a mounted on the side opposite to the first side of the body portion 1 or a side adjacent to the opposite side and a second electrode unit 24b mounted on the first side of the body portion 1. The first electrode unit 24a may include at least one electrode (e.g., a measurement electrode) and is fixed at a position exposed to the outside when the body portion 1 is inserted in and worn on the outer ear. The second electrode unit 24b includes at least two or more electrodes (e.g., a ground electrode, a reference electrode, etc.), and may be in contact with the wearer's ear skin or close to the ear skin when the body portion 1 is inserted in and worn on the outer ear.

The PPG sensor 26 is preferably close to the wearer's skin.

FIG. 2 is a block diagram of a wireless sound converting system having a bio-signal detection function according to the present disclosure.

A wireless sound converting system includes a pair of wireless sound converting devices 10a and 10b performing wireless communication with an electronic communication device 30 and performing a sound reproduction function, a phone call function, and a bio-signal detection function, etc. and the electronic communication device performing wireless communication with at least one wireless sound converting device 10a and 10b and allowing the wireless sound converting device 10 to perform a sound reproduction function, a phone call function, and a bio-signal detection function, etc.

Each of the wireless sound converting devices 10a and 10b has the same mechanical configuration as the wireless sound converting device 10 in FIG. 1A.

The wireless sound converting device 10a includes an input unit 11a obtaining an input (e.g., power on/off, operation of a bio-signal detection function and stopping of the operation, selection of a normal mode and a precision mode, wireless communication connection and termination of connection, etc.) from a user and applying the acquired input to a data processor 29a, a display unit 13a displaying various information (e.g., power status, whether a bio-signal detection function is performed, physical condition, etc.), a microphone 15a acquiring sound and applying the acquired sound to a data processor 29a, a speaker 17a emitting sound through the sound passage 3a based on an electrical signal from the data processor 29a, and first and second optical elements 18a and 18b. The wireless sound converting device 10a further includes a light irradiating unit 19a performing optical irradiation according to a control signal from the data processor 29a, a communication unit 21a performing wireless communication with the electronic communication device 30 and/or the wireless sound converting device 10b, and a power supply unit 23a supplying power. The wireless sound converting device 10a further includes an electrocardiogram (ECG) sensor 25a detecting an ECG detection value (hereinafter, a first detection value) corresponding to ECG of the wearer and applying the detected ECG detection value to the data processor 29a, a PPG sensor 26a detecting a detection value (hereinafter, a second detection value) for the PPG of the wearer and applying the detected detection value to the data processor 29a, an inertial measurement unit (IMU) sensor 28a detecting a detection value (hereinafter, a third detection value) on a change of inertia of the wireless sound converting device 10a and applying the detected detection value to the data processor 29a, and a data processor 29a controlling the aforementioned components and performing communication with the electronic communication device 30 to perform a sound reproduction function, a phone call function, and a bio-signal detection function.

In addition, a control device including the input unit 11a, the display unit 13a, the microphone 15a, the speaker 17a, the light irradiating unit 19a, the communication unit 21a, the power supply unit 23a, the ECG sensor 25a, the PPG sensor 26a, the IMU sensor 28a, and the data processor 29a is mounted in the accommodating space or side surfaces of the body portion 1.

However, the input unit 11a, the display unit 13a, the microphone 15a, the speaker 17a, the communication unit 21a, and the power supply unit 23a are naturally known to a person skilled in the art to which the present disclosure pertains, and thus, a detailed description thereof will be omitted.

The light irradiating unit 19a is controlled by the data processor 29 and includes a plurality of first and second optical devices 18a and 18b for irradiating light in visible and near-infrared bands in the range of 650 nm to 1,300 nm. The light irradiating unit 19a may include LED devices, for example.

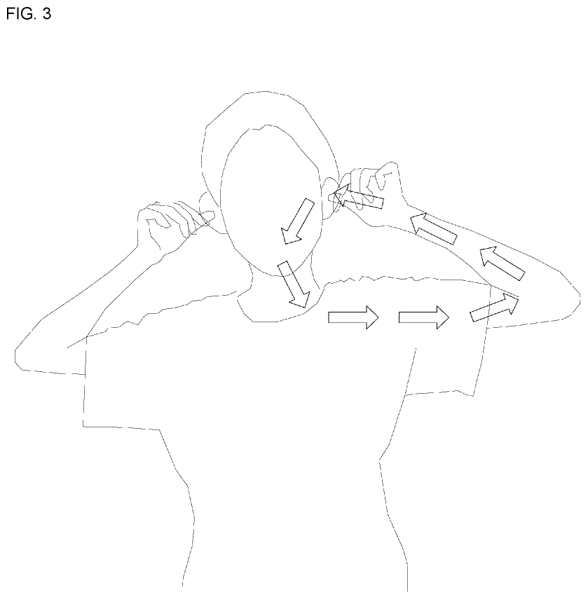

The ECG sensor 25a acquires a first detection value, which is ECG as a result of continuous electric polarization action of an atrial muscle, through the electrode unit 24 in contact with a human body surface and applies the obtained first detection value to the data processor 29a. In order for the electrode unit 24 to contact the wearer's skin, the first electrode unit 24a and the second electrode unit 24b have to contact the skin. Since a bio-signal transmission path has to be formed by such skin contact, in the present disclosure, formation of the bio-signal transmission path is guided as shown in FIG. 3, which will be described in detail below.

The data processor 29a stores a reference ECG value for determining abnormality of hypersthenia (heart muscle) or hypersthenia (abnormality of excitement). The data processor 29a determines a physical condition of the wearer by comparing the first detection value with the reference ECG value. For example, if the first detection value exceeds the reference ECG value, the data processor 29a determines that the wearer's physical condition is abnormal, otherwise the data processor 29a determines that the wearer's physical condition is normal.

The PPG sensor 26a includes a light emitting portion and a light receiving portion. By irradiating light to the wearer's skin by the light emitting portion and measuring light reflected from the wearer's skin by the light receiving portion, a second detection value, which is a detection value for PPG, is measured and applied to the data processor 29a.

The data processor 29a receives a second detection value and generates a physical condition index (e.g., heart rate, blood flow rate, blood pressure, oxygen saturation, etc.) for checking the physical condition of the wearer from the second detection value. In addition, the data processor 29a stores a reference index corresponding to the physical condition index for determining the physical condition of the wearer. The data processor 29a compares the physical condition index with the reference index to determine the physical condition of the wearer. For example, if the physical condition index exceeds the reference index, the data processor 29a may determine that the wearer's physical condition is abnormal, otherwise, the wearer's physical condition may be determined as normal. However, the second detection value by the PPG sensor 6 may be measured at all times without an additional operation of the wearer (e.g., the formation of the bio-signal transmission path in FIG. 3), but reliability thereof is known to be lower than reliability of the first detection value from the ECG sensor 25. In the normal mode below, the data processor 29a determines the physical condition of the wearer using the second detection value, and in order to supplement the relatively low reliability, in the present embodiment, a precision mode below is performed as described hereinafter.

In addition, the IMU sensor 28a detects a third detection value, which is a change in inertia, and applies the detected third detection value to the data processor 29a. The IMU sensor 26a includes an acceleration sensor that detects movement in three axes of front and back, up and down, and left and right in a three-dimensional space and a gyroscope sensor that detects three-axis rotation of pitch, roll, and yaw. The IMU sensor 26a detects the third detection value and applies the detected third detection value to the data processor 29a.

The data processor 29a may improve accuracy of the physical condition index corresponding to the first detection value using the third detection value. Even in the same physical condition of the wearer, for example, a heart rate detected by the PPG sensor 26a, for example, may be varied by a change in measurement position or movement of the PPG sensor 26a. In consideration of this, when calculating the physical condition index by processing the first detection value, the data processor 29a may correct the physical condition index in consideration of the third detection value or calculate the physical condition index by correcting the first detection value, thereby improving accuracy of the physical condition index

In addition, the ECG sensor 25a may perform precise measurement, but since electrical distortion may be included in the first detection value, it may be difficult to determine the physical condition of the wearer. However, since the data processor 29a corrects distortion of an electrical signal of the first detection value using the second detection value from the PPG sensor 26a, the physical condition of the wearer may be determined using the more accurate first detection value. The process of correcting the first detection value using the second detection value may be implemented with various algorithms, and this technology is known to a person skilled in the art to which the present disclosure pertains, and thus, a detailed description thereof will be omitted.

The data processor 29a may perform an already well-known sound reproduction function and a phone call function and may include a processor (e.g., a CPU, microprocessor, MCU, etc.) performing a bio-signal detection function using at least one of the ECG sensor 25a, the PPG sensor 26a, and the IMU sensor 28a and a storage unit (e.g., memory, etc.) storing the first to third detection values, the reference PPG value, the physical condition index, the reference index, etc. The bio-signal detection function will be described in detail below.

The wireless sound converting device 10b has the same configuration as the wireless sound converting device 10a, but may be distinguished according to a communication control method. A case in which the data processor 29a and the data processor 29b control the communication units 21a and 21b, respectively, to independently perform communication with the electronic communication device 30 and a case in which the data processor 29a controls the communication unit 21a to communicate with the electronic communication device 30 and the data processor 29b controls the communication unit 21b to communicate with the communication unit 21a of the data processor 29a (in which the wireless sound converting device 10a serves as a master device and the wireless sound converting device 10b serves as a slave device that is communicatively connected to and controlled by the wireless sound converting device 10a). The communication control method between the wireless sound converting devices 10a and 10b and the electronic communication device 30 corresponds to a technology already known to a skilled person in the art to which the present disclosure pertains, and thus, a detailed description thereof will be omitted. However, in the embodiment, the wireless sound converting device 10 is collectively referred to as the wireless sound converting devices 10a and 10b, and the communication method therebetween corresponds to any one of the aforementioned cases.

In addition, the ECG sensor 25 (collectively referred to as 25a and 25b), the PPG sensor 26 (collectively referred to as 26a and 26b), and the IMU sensor 28 (collectively referred to as 28a and 28b) should be understood that they may be mounted on at least one of the wireless sound converting devices 10a and 10b. In this case, it is preferable that the PPG sensor 26 and the IMU sensor 28 are mounted together in the same wireless sound converting device 10.

The electronic communication device 30 corresponds to, for example, an electronic communication device such as a smartphone, a tablet, etc., and includes an input unit 31 acquiring an input (e.g., operation of the bio-signal detection function and stopping of the operation, selection of the normal mode and the precision mode, selection of an independent mode and a control mode, etc.) from the user and applying the acquired input to a data processor 39, a display unit 33 visually and/or audibly displaying an operation of the bio-signal detection function, the stopping of the operation, the normal mode and the precision mode, and the user's physical condition, etc., a communication unit 35 performing wireless communication with the wireless sound converting device 10, and a data processor 39 performing the widely known phone call function and the sound reproduction function and the bio-signal detection function described below. However, a power supply unit (not shown), the input unit 31, the display unit 33, and the communication unit 35 are known to a person skilled in the art to which the present disclosure pertains, and thus a detailed description thereof will be omitted.

The data processor 39 performs communication with the wireless sound converting device 10 to perform a phone call function and a sound reproduction function, and includes a processor (e.g., a CPU, a microprocessor, an MCU, etc.) performing a bio-signal detection function using the first to third detection values from each of the ECG sensor 25, the PPG sensor 26, and the IMU sensor 28 described above and a storage unit (e.g., memory, etc.) storing the first to third detection values, a reference PPG value, a physical condition index, a reference index, and the like. The bio-signal detection function will be described in detail below.

The bio-signal detection function may be performed while at least one of the wireless sound converting devices 10a and 10b is worn on the wearer's ear.

In this embodiment, the normal mode of the bio-signal detection function calculates a physical condition index based on the second detection value from the PPG sensor 26, and compares the physical condition index with the reference index to determine the wearer's physical condition.

The precise mode of the bio-signal detection function is a mode of determining the wearer's physical condition using at least two or more of the first to third detection values from the ECG sensor 25, the PPG sensor 26, and the IMU sensor 28 described above.

In addition, the wireless sound converting device 10 may independently perform the bio-signal detection function without control of the electronic communication device 30 (this is referred to as “independent mode”), and the electronic communication device 30 may control the wireless sound converting device 10 to perform the bio-signal detection function (this is referred to as a “control mode”). The normal mode and the precision mode may be performed in the independent mode, and the normal mode and the precision mode may be performed in the control mode as well.

First, the normal mode in the independent mode is described. The data processor 29 (collectively referred to as 29a and 29b) calculates a physical condition index from the second detection value from the PPG sensor 26 provided in the wireless sound converting device 10, and compares the physical condition index with the reference index to determine the wearer's physical condition. The data processor 29 may determine the physical condition of the wearer more accurately using the third detection value from the IMU sensor 28 as described above, during or before the calculation of the physical condition index. The data processor 29 may inform the wearer of the determined physical condition and/or physical condition index through the display unit 13 (collectively referred to as 13a and 13b) or the speaker 17 (collectively referred to as 17a and 17b). In addition, the data processor 29 may transmit the determined physical condition and/or the physical condition index to the electronic communication device 30 through the communication unit 21 (collectively referred to as 21a and 21b), and the data processor 39 may receive the physical condition and/or the physical condition index through the communication unit 35 and display the received physical condition and/or the physical condition index through the display unit 33 for wearer's information.

Next, the precision mode in the independent mode is described. The data processor 29 may calculate more precise physical condition index (e.g., heart rate (HR), heart rate variability (HRV), blood pressure, etc.) using the first detection value from the ECG sensor 25 and the second detection value from the PPG sensor 26 and compare the calculated physical condition index with the reference index to determine the wearer's physical condition. The data processor 29 may determine the physical condition of the wearer more accurately using the third detection value from the IMU sensor 28 as described above, during or before the calculation of the physical condition index. The data processor 29 may inform the wearer of the determined physical condition and/or physical condition index through the display unit 13 (collectively referred to as 13a and 13b) or the speaker 17 (collectively referred to as 17a and 17b). In addition, the data processor 29 may transmit the determined physical condition and/or the physical condition index to the electronic communication device 30 through the communication unit 21 (collectively referred to as 21a and 21b), and the data processor 39 may receive the physical condition and/or the physical condition index through the communication unit 35 and display the received physical condition and/or the physical condition index through the display unit 33 for wearer's information.

In the above precision mode, in order for the ECG sensor 25 to detect the first detection value, a bio-signal transmission path should be configured between the heart and the wireless sound converting device 10, and the bio-signal transmission path is illustrated in FIG. 3 in detail.

In addition, the data processor 29 may automatically perform the precision mode when the wearer's physical condition is abnormal while performing the normal mode of the independent mode, or, as described above, the data processor 29 may inform that the wearer's physical condition is abnormal, inform the wearer as to whether the precision mode is to be performed through the speaker 17, acquire an instruction for performing the precision mode from the input unit 11, and subsequently perform the precision mode.

Next, the normal mode in the control mode is described. The data processor 39 transmits a normal mode operation command to the wireless sound converting device 10 communicatively connected through the communication unit 35 according to a normal mode selection input from the input unit 31. The data processor 29 controls the PPG sensor 26 and the IMU sensor 28 according to the operation command, calculates a physical condition index from the second detection value from the PPG sensor 26 provided in the wireless sound converting device 10, and compares the physical condition index with the reference index to determine a physical condition of the wearer. The data processor 29 may more accurately determine the wearer's physical condition using the third detection value from the IMU sensor 28 as described above, during or before the calculation of the physical condition index. In addition, the data processor 29 may transmit the determined physical condition and/or the physical condition index to the electronic communication device 30 through the communication unit 21 (collectively referred to as 21a and 21b), and the data processor 39 may receive the physical condition and/or the physical condition index through the communication unit 35 and display the received physical condition and/or the physical condition index through the display unit 33 for wearer's information.

Alternatively, in the normal mode of the control mode, the data processor 29 may transmit the second detection value and the third detection value to the electronic communication device 30 through the communication unit 21, and the data processor 39 may determine the wearer's physical condition as performed by the data processor 29 described above using the second value and the third detection value, and display the determined physical condition and the physical condition index through the display unit 33.

Next, the precision mode of the control mode is described. The data processor 39 transmits an operation command of the precision mode to the wireless sound converting device 10 communicatively connected through the communication unit 35 according to a precision mode selection input from the input unit 31. The data processor 29 receives the operation command through the communication unit 21, and calculates more precise physical condition index (e.g., heart rate (HR), heart rate variability (HRV), blood pressure, etc.) using the first detection value from the ECG sensor 25 and the second detection value from the PPG sensor 26 according to the operation command and compare the calculated physical condition index with the reference index to determine the wearer's physical condition. The data processor 29 may determine more accurately the wearer's physical condition using the third detection value from the IMU sensor 28 as described above during or before the calculation of the physical condition index. The data processor 29 may inform the wearer of the determined physical condition and/or physical condition index through the display unit 13 or the speaker 17. In addition, the data processor 29 may transmit the determined physical condition and/or physical condition index to the electronic communication device 30 through the communication unit 21, and the data processor 39 may receive the physical condition and/or physical condition index through the communication unit 35 and inform the wearer through the display unit 33 accordingly.

Alternatively, in the precision mode of the control mode, the data processor 29 may transmit the first to third detection values to the electronic communication device 30 through the communication unit 21, and the data processor 39 may determine the wearer's physical condition as performed by the data processor 29 described above using the first to third detection values, and display the determined physical condition and physical condition index through the display unit 33.

In addition, while performing the normal mode of the control mode, the data processor 39 may automatically perform the precision mode if the wearer's physical condition is abnormal, or, as described above, the data processor 29 may inform that the wearer's physical condition is abnormal, inform the wearer as to whether the precision mode is to be performed through the display unit 33, acquire an instruction for performing the precision mode from the input unit 11, and subsequently perform the precision mode.

FIG. 3 is a schematic diagram of configuring a bio-signal transmission path for detection of a first detection value of an ECG sensor.

As shown in FIG. 3, in a state in which the wireless sound converting device 10 equipped with the ECG sensor 25 is worn on the wearer's ear, the wearer may contact or press the first electrode unit 24a of the wireless sound converting device 10 with his or her hand or finger. The first electrode unit 24a may then be in contact with the wearer's hand or finger and contact an ear skin to form a bio-signal transmission path. Since the heart is located in the left chest, the bio-signal transmission path is preferably configured such that the wireless sound converting device 10 is worn on the left ear of the wearer and a left hand contacts the first electrode unit 24a.

To configure such a bio-signal transmission path, in the precision mode of the independent mode, the data processor 29 emits sound of a message (e.g., Please bring a hand in the direction in which the wireless sound converting device is worn into contact with the first electrode unit of the wireless sound converting device and press the first electrode unit) to configure the bio-signal transmission path. Alternatively, in the precision mode of the control mode, the data processor 39 transmits the message for configuring the bio-signal transmission path to the wireless sound converting device 10 through the communication unit 35, and the data processor 29 emits sound of the message for configuring the bio-signal transmission path through the speaker 17.

That is, the data processor 29 proceeds to guide the configuration or formation of the bio-signal transmission path as described above to acquire the first detection value by the ECG sensor 25.

In addition, the data processor 29 controls the ECG sensor 25 to receive a first detection value from the ECG sensor 25, and if the received first detection value is a detection value when a normal bio-signal transmission path is configured, the data processor 29 determines that the bio-signal transmission path is configured, and if not, the data processor 29 repeatedly emits sound of a message for configuring the bio-signal transmission path through the speaker 17.

At least a part of devices (e.g., modules or functions thereof) or methods (e.g., operations) according to various embodiments of the present disclosure may be implemented as instructions stored in a computer-readable storage medium in the form of a program module. In a case where the instructions are performed by a processor, one or more processors may perform functions corresponding to the instructions. The computer-readable storage medium may be, for example, a memory.

A computer-readable recording medium may include a hard disk, a floppy disk, a magnetic medium (e.g., a magnetic tape), an optical medium (e.g., CD-ROM, digital versatile disc (DVD)), a magneto-optical medium (e.g., a floptical disk), or a hardware device (e.g., a ROM, a RAM, a flash memory, or the like). The program instructions may include machine language codes generated by compilers and high-level language codes that may be executed by computers using interpreters. The above-mentioned hardware device may be configured to be operated as one or more software modules for performing operations of various embodiments of the present disclosure and vice versa.

A processor or a functions based on the processor according to various embodiments of the present disclosure may include at least one of the above-mentioned elements, or some elements may be omitted or other additional elements may be added. Operations performed by the module, the program module or other elements according to various embodiments of the present disclosure may be performed in a sequential, parallel, iterative or heuristic way. Furthermore, some operations may be performed in another order or may be omitted, or other operations may be added.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A wireless sound converting system, comprising:
a bio-signal detection function including first and second wireless sound converting devices including a body portion having an accommodation space formed therein, an ear tip mounted on a first side of the body portion and having a sound passage, a speaker configured to emit sound through a sound path, a microphone formed in the body portion, a communication unit configured to perform communication with an electronic communication device, and a data processor configured to perform a sound reproduction function by controlling the communication unit to communicate with the electronic communication device,
wherein at least one of the first and second wireless sound converting devices includes: an internal electrode unit mounted on the first side of the body portion and configured to detect electrocardiogram (ECG), and an external electrode unit mounted on a side opposite to the first side or a side adjacent to the opposite side and including an ECG sensor configured to apply a first detection value related to ECG to the data processor and a photoplethysmography (PPG) sensor mounted on the first side of the body portion and configured to detect PPG and apply a second detection value related to PPG to the data processor,
wherein in a state in which the first or second wireless sound converting device including the ECG sensor is mounted on a wearer's ear, the data processor of the mounted wireless sound converting device is configured to emit sound of a message for configuring a bio-signal transmission path using the wearer's body through the speaker to measure ECG.

2. The wireless sound converting system of claim 1, wherein at least one wireless sound converting device including the PPG sensor, among the first and second wireless sound converting devices, includes an inertial measurement unit (IMU) sensor configured to apply a third detection value for a change in inertia to the data processor.

3. The wireless sound converting system of claim 2, wherein any one or more of the data processors of the first and second wireless sound converting devices is configured to transmit at least one of the first to third detection values to the electronic communication device through the communication unit or determine a physical condition of the wearer who wears the first or second wireless sound converting device using at least one of the first detection value and the second detection value, and notify the determined physical condition through the speaker.

4. The wireless sound converting system of claim 3, wherein:
the wireless sound converting system includes an electronic communication device configured to receive at least one or more of the first and second detection values from at least one or more of the first and second wireless sound converting devices; and
the electronic communication device is configured to determine the physical condition of the wearer using at least one or more of the first and second detection values, and transmit the determined physical condition to at least one or more of the first and second wireless sound converting devices.

5. The wireless sound converting system of claim 1, wherein the data processor of the mounted wireless sound converting device is configured to emit, through the speaker, sound of a message for configuring a bio-signal transmission path that allows the wearer to press and contact the outer electrode unit with a hand or a finger.

6. The wireless sound converting system of claim 1, wherein any one or more of the data processors of the first and second wireless sound converting devices is configured to transmit at least one of the first and second detection values to the electronic communication device through the communication unit or determine a physical condition of the wearer who wears the first or second wireless sound converting device using at least one of the first detection value and the second detection value, and notify the determined physical condition through the speaker.

7. The wireless sound converting system of claim 6, wherein any one or more of the data processors of the first and second wireless sound converting devices is configured to determine the physical condition of the wearer using the second detection value and the third detection value.

8. The wireless sound converting system of claim 6, wherein:
the wireless sound converting system includes an electronic communication device configured to receive at least one or more of the first to third detection values from at least one or more of the first and second wireless sound converting devices; and
the electronic communication device is configured to determine the physical condition of the wearer using at least one or more of the first to third detection values, and transmit the determined physical condition to at least one or more of the first and second wireless sound converting devices.
